(19) United States
(12) Patent Application Publication
(10) Pub. No.: US 2009/0291118 A1
(43) Pub. Date: Nov. 26, 2009

(54) UNIVERSAL BARRIER TO PREVENT INFECTIONS FROM HUMAN IMMUNODEFICIENCY VIRUS

(76) Inventors: Lane Bernard Scheiber, Annandale, VA (US); Lane Bernard Scheiber, II, Grosse Ile, MI (US)

Correspondence Address: LANE B. SCHEIBER, 7864 OAK RIVER DRIVE, GROSS ILE, MI, 48138, US

(21) Appl. No.: 12/126,901

(22) Filed: May 25, 2008

(51) Int. Cl. A61K 9/00; A61P 31/18

(52) U.S. Cl. 424/430; 424/435; 424/436

(57) ABSTRACT

The Human Immunodeficiency Virus poses a significant threat to the world's population. Current strategies to treat infectious agents have not been adequate to eradicate such deadly viral infections. HIV seeks out its host, a T-Helper cell, by utilizing glycoprotein 120 probes to engage a CD4 cell-surface receptor located on the surface of a T-Helper cell. Developing devices to offer HIV virions' probes the opportunity to engage the cell-surface receptors they are seeking offers a means of neutralizing the infectious threat of HIV. A device in the form of a solution containing a filter medium comprised of sheets or strips or spheres of lipid bilayer or virus-like structures or hypoallergenic surfaces to carry cell-surface receptors, each type of medium having affixed to its surface cell-surface receptors intended to engage and neutralize the infectious nature of HIV virions provides an effective strategy to avert AIDS.

### BACKGROUND OF THE INVENTION

1. Field of the Invention

This invention relates to any medical device that is utilized to act as a barrier to protect a person from contracting the Human Immunodeficiency Virus by neutralizing the infectious nature of Human Immunodeficiency Virus virions.

2. Description of Background Art

It has been estimated by the Center for Disease Control that in the United States 55,000 to 60,000 new cases of Human Immunodeficiency Virus (HIV) are occurring each year. It is thought that there are 900,000 people currently infected with HIV in the United States, with many victims not aware that they have contracted the virus. Further, it has been estimated that the Human Immunodeficiency Virus (HIV), the pathogen that causes Acquired Immune Deficiency Syndrome (AIDS), has infected as many as 30-60 million people around the globe.

The presence of HIV was first came to the attention of those in the United States in 1981, when there appeared an outbreak of Kaposi's Sarcoma and Pneumocystis carinii pneumonia in gay men in New York and California. After over twenty-five years of research and investigation, eradicating the ever growing global humanitarian crisis posed by the HIV remains an elusive goal for the medical community. It is estimated the virus has already killed 25 million citizens of this planet.

The Human Immunodeficiency Virus has been previously referred to as human T-Lymphotrophic virus III (HTLV-III), lymphadenopathy-associated virus (LAV), and AIDS-associated retrovirus (ARV). Infection with HIV may occur by the virus being transferred by blood, semen, vaginal fluid, or breast milk. Four major means of transmission of HIV include unprotected sexual intercourse, contaminated needles, breast milk, and transmission from an infected mother to her baby at birth.

HIV is an ingeniously constructed very deadly virus, which represents the most challenging pathogen the worldwide medical community faces to date. Viruses in general, have been difficult to contain and eradicate due to the fact they are obligate parasites and tend not to carry out any biologic functions outside the cell the virus has targeted as its host. A virus when it exists outside the boundaries of a cell is generally referred to as a virion. HIV virions posses several attributes that make them very elusive and difficult to destroy.

For purposes of this text, the term ‘body’ refers to the material part of a man or a woman, generally including the head, neck, trunk, extremities and all usual internal structures. For the purposes of this text, the term ‘vagina’ refers to the genital canal in a woman extending from the uterus to the vulva.

Bacterial infections have posed an easier target for the medical community to eradicate from the body. Bacteria generally live and reproduce outside animal cells. Bacteria, like animal cells, carryout biologic functions. A large multi-celled organism such as the human body combats bacterial infections with a combined force of white cells, antibodies, complements and its lymphatic system. White cells circulate the body in search of bacteria. When a white cell encounters a bacterium, the white cell engulfs the bacterium, encapsulates the pathogen, processes the identification of the pathogen and kills the pathogen utilizing acids and destructive enzymes. The white cell then alerts the B-cells of the immune system as to the identity of the intruding bacterium. A subpopulation of B-cells is generated, dedicated to producing antibodies directed against the particular pathogen the circulating white cell encountered and identified. Antibodies, generated by B-cells, traverse the blood and body tissues in search of the bacteria they were designed to repel. Once an antibody encounters a bacterium it is targeted to attack, the antibody attaches to the bacterium's outer wall. The effect antibodies have in coating the outside of a bacterium is to assist the white cells and the other components of the immune system in recognizing the bacterium, so that appropriate defensive action can be taken against the pathogen. Some antibodies, in addition to coating the bacterium, will act to punch holes through the bacterium's outer wall. If the integrity of the bacterium's cell wall is breached, this action generally leads to the death of the bacterium. Complements are primitive protein structures that circulate the blood stream in search of anything that appears consistent with a bacteria cell wall. Complements are indiscriminant. Once the complement proteins locate any form of bacterial cell wall, the complement proteins organize, and much like antibodies, act in concert to punch one or more holes though a bacterium's cell wall to compromise the viability of the bacterium. The lymphatic system is a diffuse network of thin walled vessels that drain excess water from extracellular fluids and join to form the thoracic duct and right lymph duct, which empty into the venous system near the heart. Lymph nodes are present at different locations in the body and screen the fluid transiting the lymphatic system, called lymph, to remove pathogens. Cells in the spleen screen the blood in search of bacteria. When a bacterial pathogen is identified, such as by antibodies coating the surface, the bacterium is taken out of circulation and terminated.

Viruses pose a much different infectious vector to the body's defense system than either bacteria or cellular parasites. Since viruses do not carry out biologic processes outside their host cell, a virus can be destroyed, but they cannot be killed. A virus is simply comprised of one or more external shells and a portion of genetic material. The virus's genetic information is carried in the core of the virus. Antibodies can coat the exterior of a virus to make it easier for the white cells in the body to identify the viral pathogen, but the action of punching holes in the virus's external shell by antibodies or complement proteins does not necessarily kill the virus. Viruses also only briefly circulate in the blood and tissues of the body as an exposed entity. Using exterior probes, a virus hunts down a cell in the body that will act as an appropriate host so that the virus can replicate. Once the virus has found a proper host cell, the virus inserts its genome into the host cell. To complete its life-cycle, the virus's genetic material takes command of cellular functions and directs the host cell to make replicas of the virus.

Once the virus's genome has entered a host cell, the virus is in effect shielded from the body's immune system defense mechanisms. Inside a host cell, the presence of the virus is generally only represented as genetic information incorporated into the host cell's DNA. Once a virus has infected a cell in the body, the presence of the virus can only be eradicated if the host cell is destroyed. Antibodies and complements are generally designed not to attack the autologous tissues of the body. Circulating white cells and the immune cells which comprise lymph nodes and the spleen may or may not recognize that a cell, which has become a host for a virus, is infected with a virus's genome. If the immune system fails to identify a cell that has become infected with a virus, the virus's genetic material can proceed to force the infected cell to make copies of the virus. Since a virus is in essence simply a segment of genetic material, time is of no consequence to the life-cycle of the virus and a virus's genome may be carried for years by the host without a need to activate; such viruses are often termed latent viruses. A virus's genetic material may sit idle in a host cell for an extended period of time until the pathogen's programming senses the time is right to initiate the virus's replication process or an action of the host cell triggers the virus to replicate. The only opportunity for the immune system to destroy a latent virus is when copies of the virus leave the host cell and circulate in the blood or tissues in search of another perspective host cell.

The traditional medical approach to combating infectious agents such as bacteria and cellular parasites, therefore has limited value in managing or eradicating elusive or latent viral infections. Synthetic antibiotics, generally used to augment the body's capacity to produce naturally occurring antibodies against bacterial infections, have little success in combating latent viral infections. Stimulating the body's immune system's recognition of a virus by administering a vaccine also has had limited success in combating elusive viral infections. Vaccines generally are intended to introduce to the body pieces of a bacteria or virus, or an attenuated, noninfectious intact bacteria or virus so that the immune system is able to recognize and process the infectious agent and generate antibodies directed to assist in killing the pathogen. Once the immune system has been primed to recognize an intruder, antibodies will be produced by the immune system in great quantities in an effort to repel an invader. Over time, as the immune system down-regulates its antibody production in response to a lack of detecting the presence of the intruding pathogen, the quantity of antibodies circulating in the blood stream may decrease in number to a quantity that is insufficient to combat a pathogen. Since antibodies have limited value in combating some of the more elusive viruses that hibernate in host cells, vaccines have limited value in destroying latent viruses.

The Human Immunodeficiency Virus demonstrates four factors which make this pathogen particularly elusive and a difficult infectious agent to eradicate from the body. First: the host for HIV is the T-Helper cell. The T-Helper cell is a key element in the immune system's response since it helps coordinate the body's defensive actions against pathogens seeking to invade the body's tissues. In cases of a bacterial infection versus a viral infection, T-Helper cells actively direct which immune cells will rev-up in response to the infectious agent and engage the particular pathogen. Since HIV infects and disrupts T-Helper cells, coordination of the immune response against the virus is disrupted, thus limiting the body's capacity to mount a proper response against the presence of the virus and produce a sufficient action to successfully eradicate the virus.

Second: again, latent viruses such as HIV, have a strategic advantage. When the immune system first recognizes a pathogen and begins to generate antibodies against a particular pathogen, the response is generally robust. Once time has passed and the immune system fails to detect an active threat, the production of antibodies against the particular pathogen diminishes. When HIV infects a T-Helper cell, the viral genome may lay dormant, sometimes for years before taking command of the T-Helper cell's biologic functions. HIV may, therefore, generate a very active initial immune response to its presence, but if the virus sits dormant inside T-Helper cells for months or years, the antibody response to the virus will diminish over time. There may not be an adequate quantity of circulating antibodies to actively engage the HIV virions as they migrate from the T-Helper cell that generated the copies to uninfected T-Helper cells that will serve as a new host to support further replication. If the immune system's response is insufficient during the period while the virus is exposed and vulnerable, it becomes extremely difficult for the body to eradicate the virus.

Third, when replicas of the Human Immunodeficiency Virus are released from their host cell, during the budding process the HIV virion coats itself with an exterior envelope comprised of a portion of the plasma membrane from the T-Helper cell that acted as the host for the virus. A T-Helper cell's plasma membrane is comprised of a lipid bilayer, a double layer of lipid molecules oriented with their polar ends at the outside of the membrane and the nonpolar ends in the membrane interior. The virus thus, in part, takes on an external appearance of a naturally occurring cell in the body. Since the exterior envelope of a HIV virion has the characteristics of a T-Helper cell it is more difficult for the immune system to recognize that it is a pathogen as it migrates through the body in search of another T-Helper cell to infect.

Fourth, the Human Immunodeficiency Virus exhibits a very elusive mode of action which the virus readily utilizes to actively defeat the body's immune system. HIV carries in its genome a segment of genetic material that directs an infected T-Helper cell to create and mount on the surface the plasma membrane a FasL cell-surface receptor. Healthy T-Helper cells carry on the surface of their plasma membrane Fas cell-surface receptors. The Fas cell-surface receptor when engaged by a FasL cell-surface receptor on another cell, initiates apoptosis in the cell carrying the Fas cell-surface receptor. Apoptosis is a biologic process that causes a cell to terminate itself. A T-Helper cell infected with the HIV virus carrying a FasL cell-surface receptor is therefore capable of killing noninfected T-Helper cells that the infected T-Helper cell encounters as it circulates the body. The occurrence of AIDS is therefore propagated not only by the number of T-Helper cells that become incapacitated due to direct infection by HIV, but also by the number of noninfected T-Helper cells that are eliminated by coming in direct contact with infected T-Helper cells.

Acquired Immune Deficiency Syndrome (AIDS) occurs as a result of the number of circulating T-Helper cells declining to a point where the immune system's capacity to mount a successful response against opportunistic infectious agents is significantly compromised. The number of viable T-Helper cells declines either because they become infected with the HIV virus or because they have been killed by encountering a T-Helper cell infected with HIV. When there is an insufficient population of non-HIV infected T-Helper cells to properly combat infectious agents such as Pneumocystis carinii or cytomegalo virus or other pathogens, the body becomes overwhelmed with the opportunistic infection and the patient becomes clinically ill. In cases where the combination of the patient's compromised immune system and medical assistance in terms of synthetic antibiotics intended to combat the opportunistic pathogens, fluids, intravenous nutrition and other treatments are not sufficient to sustain life, the body succumbs to the opportunistic infection and death ensues.

The Human Immunodeficiency Virus locates its host by utilizing probes located on its envelope. The HIV virion has two types of glycoprotein probes attached to the outer surface of its exterior envelope. A glycoprotein is a structure comprised of a protein component and a lipid component. HIV utilizes a glycoprotein 120 (gp 120) probe to locate a CD4 cell-surface receptor on the plasma membrane of a T-Helper cell. The plasma membrane of the T-Helper cell is comprised of a lipid bilayer. Cell-surface receptors are anchored in the lipid bilayer. Once an HIV gp 120 probe has successfully engaged a CD4 cell-surface receptor on a T-Helper cell a conformational change occurs in the gp 120 probe and a glycoprotein 41 (gp 41) probe is exposed. The gp 41 probe's intent is to engage a CXCR4 or CCR5 cell-surface receptor on the plasma membrane of the same T-Helper cell. Once a gp 41 probe on the HIV virion engages a CXCR4 or CCR5 cell-surface receptor, the HIV virion opens an access portal through the T-Helper cell's plasma membrane.

Once the virus has gained access to the T-Helper cell by opening a portal through the cell's outer membrane the virion inserts two positive strand RNA molecules approximately 9500 nucleotides in length. Inserted along with the RNA strands are the enzymes reverse transcriptase, protease and integrase. Once the virus's genome gains access to the interior of the T-Helper cell, in the cytoplasm the pair of RNA molecules are transformed to deoxyribonucleic acid by the reverse transcriptase enzyme. Following modification of the virus's genome to DNA, the virus's genetic information migrates to the host cell's nucleus. In the nucleus, with the assistance of the integrase protein, the virus's DNA becomes inserted into the T-Helper cell's native DNA. When the timing is appropriate, the now integrated viral DNA, becomes read by the host cell's polymerase molecules and the virus's genetic information commands certain cell functions to carry out the replication process to construct copies of the human deficiency virus.

Present anti-viral therapy has been designed to target the enzymes that assist the HIV genome with the replication process. Anti-viral therapy is intended to interfere with the action of these replication enzymes. Part of the challenge of eradicating HIV is that once the virus inserts its genome into a T-Helper cell host, the viral genome may lay dormant until the proper circumstances evolve. The virus's genome may sit idle inside a T-Helper cell for years before becoming activated, causing drugs that interfere with HIV's life cycle to have limited effect on eliminating the virus from the body. Arresting the replication process does not insure that T-Helper cells infected with HIV do not continue to circulate the body killing noninfected T-Helper cells thus causing the patient to progress to a clinically apparent state of Acquired Immune Deficiency Syndrome and eventually succumbing to an opportunistic infection which eventually results in the death of the individual.

The outer layer of the HIV virion is comprised of a portion of the T-Helper cell's outer cell membrane. In the final stage of the replication process, as a copy of the HIV capsid, carrying the HIV genome, buds through the host cell's plasma membrane, the capsid acquires as its outermost shell a wrapping of lipid bilayer from the host cell's plasma membrane. Vaccines are generally comprised of pieces of a virus or bacterium, or copies of the entire virus or bacterium weakened to the point the pathogen is incapable of causing an infection. These pieces of a pathogen or copies of a nonvirulent pathogen prime the immune system such that a vaccine intent is to cause B-cells to produce antibodies that are programmed to seek out the surface characteristics of the pathogen comprising the vaccine. In the case of HIV, since the surface of the pathogen is an envelope comprised of lipid bilayer taken from the host T-Helper cell's plasma membrane, a vaccine comprised of portions of the exterior envelope of the HIV virions might not only target HIV virions, but might also have deleterious effects on the T-Helper cell population. Some antibodies produced to combat HIV infections may not be able to tell the difference between an HIV virion and a T-Helper cell, and such antibodies may act to coat and assist in the elimination of both targets. In such a scenario, since such a vaccine might cause a decline in the number of available T-Helper cells, it is conceivable that a vaccine comprised of portions of the external envelope of HIV virions might paradoxically induce clinically apparent AIDS in a patient that a vaccine has been administered.

It is clear that the traditional approach of utilizing antibiotics or providing vaccines to stimulate the immune system to produce endogenous antibodies, by themselves, is an ineffective strategy to manage a virus as elusive and deadly as HIV. Drugs that interfere with the replication process of HIV generally slow progression of the infection by the virus, but do not necessarily eliminate the virus from the body nor eliminate the threat of the clinical symptoms of AIDS. A new strategy is required in order to successfully combat the threat of HIV.

The Human Immunodeficiency Virus virions are much smaller in size than the red blood cells and white blood cells. The gp120 and gp 41 probes located on the surface of HIV are seeking to engage the CD4 and CXCR4 or CCR5 cell-surface receptors located on T-Helper cells. A filter medium intended to engage HIV virions could be constructed such that body fluids which carry HIV virions would come in contact with the surface features of the filter medium. As HIV's glycoprotein probes, gp120 and gp 41, engage the CD4 and CXCR4 or CCR5 cell-surface receptors affixed to the surface of the filter medium in a similar manners as to how the CD4 and CXCR4 or CCR5 cell-surface receptors affixed to the surface of a naturally occurring T-Helper cell, the HIV virions would adhere to the filter medium and either become stuck to the filter medium or by the action of the HIV probes engaging the filter medium cell-surface receptors HIV virions would eject their genome rendering the HIV virion incapable of infecting an endogenous T-Helper cell in the woman.

The technology to make such filter medium is readably available and could be quickly implemented for worldwide use to prevent infections by HIV virions.

A device, to accomplish the task of acting as a barrier to neutralize Human Immunodeficiency Virus virions, would be to use a filter medium with constructed surface characteristics similar to the surface characteristics of a naturally occurring T-Helper cell, since, specifically, it is the cell-surface receptors affixed to the surface of a T-Helper cell that the HIV virion's probes are seeking. T-Helper cells are constructed with the outer membrane being comprised of a lipid bilayer. Cell-surface receptors are anchored into this lipid bilayer and the protein portion of the cell-surface receptor extends out and away from the surface of the T-Helper cell. Sheets of lipid bilayer constructed with a generous quantity of CD4, CXCR4 and CCR5 cell-surface receptors affixed to the surface, would attract and engage HIV virions as a surrogate target in place of endogenous T-Helper cells the HIV virions are seeking as a host.

A medical device could be constructed in a manner where a quantity of lipid bilayer fashioned in the shape of sheets or strips or spheres would be constructed with a generous quantity of CD4, CXCR4 and CCR5 cell-surface receptors affixed to the surface of the filter medium. The filter medium would be fashioned to be a liquid or a cream. As body fluids mix with the filter medium and pass across the surface of a sheet or strip or sphere of lipid bilayer, HIV virions would come in contact with CD4, CXCR4 and CXR5 cell-surface receptors present on the surface of the lipid bilayer and engage the cell-surface receptors. The HIV virions making contact with the lipid bilayer would either permanently adhere to the lipid bilayer or by engaging the cell-surface receptors on the lipid bilayer the HIV virions would be caused to eject their genome, which would neutralize the infectious threat of the HIV virions. The body fluids would be cleared of HIV virions capable of infecting T-Helper cells endogenous to a body.

Since HIV virions are searching their environment for CD4, CXCR4 and CXR5 cell-surface receptors a filter material comprised of any hypoallergenic material suitable to attach CD4, CXCR4 and CXR5 cell-surface receptors or the protein portion of these receptors, to the surface of the material. As the body fluids mix with the filter medium and pass across the surface of a sheet or strips or spheres of a hypoallergenic material with CD4, CXCR4 and CXR5 cell-surface receptors, HIV virions would come in contact with CD4, CXCR4 and CXR5 cell-surface receptors present on the surface of the hypoallergenic material and engage the cell-surface receptors. The HIV virions making contact with the cell-surface receptors affixed to the hypoallergenic material would either permanently adhere to the hypoallergenic material or by engaging the cell-surface receptors affixed to the surface of the hypoallergenic material the HIV virions would be caused to eject their genome, which would neutralize the infectious threat of the HIV virions. The body fluids would be cleared of HIV virions capable of infecting T-Helper cells endogenous to a body.

White blood cells are physically larger than red blood cells. Bacteria are generally much smaller than red blood cells. HIV virions are much smaller than bacteria. HIV is comprised of an outer envelope, an internal capsid and the viral genome. Because of its small size HIV can potentially maneuver into places in the body's tissues or crevasses between tissues where mobile cells are unable to go.

An approach to managing HIV would be to create a product that would be relatively the same size as HIV so that the product could penetrate into every location that HIV might migrate. HIV's probes are seeking the CD4 and CCR5 and CXCR4 cell-surface receptors of a T-Helper cell, thus a product to challenge HIV could be equipped with the same cell-surface receptors as would be found on a naturally occurring T-Helper cell.

Utilizing genetic machinery and a colony of T-Helper cells or a colony of hybrid T-Helper cells or a colony of specially designed host cells, a medically therapeutic modified virus or virus-like product approximately the size of a HIV virion could be manufactured in a similar manner as how HIV naturally replicates, except the product would carry the T-Helper cell cell-surface receptors CD4, CXCR4 and/or CCR5 instead of the glycoprotein probes associated with a naturally occurring HIV virion. The virus-like product would be constructed either with no genetic information present inside the capsid or genetic material to act as a filler, this genetic material being inert such that it could not carry out any useful function.

Constructing a virus-like structure, that has affixed to its exterior cell-surface receptors intended to engage a virus, is referred to as a Scientifically Modulated And Reprogrammed Target (SMART) virus. The SMART virus would be available to engage Human Immunodeficiency Virus virions present in body fluids. As HIV virions make contact with SMART viruses the HIV virions would engage the SMART virus and become permanently attached and become trapped by the filter medium, or the HIV virions, upon engaging a SMART virus, would harmlessly eject the genetic material the HIV virion carries. Either engaging and trapping the HIV virion or causing the HIV virion to eject the genetic material that it carries, would neutralize the virulence of HIV and assist in managing the threat of AIDS.

### BRIEF SUMMARY OF THE INVENTION

Initially the Human Immunodeficiency Virus is attracted to its host, the T-helper cell, by having its surface probes seek out a CD4 cell-surface receptor. Once HIV virion's gp 120 probe successfully engages a CD4 cell-surface receptor a conformation change occurs in the gp 120 probe and a gp 41 probe attempts to engage either a CXCR4 or a CCR5 cell-surface receptor located on the target T-Helper cell. Described here is a device that offers a surrogate target HIV virions are seeking. The device is intended to be applied to any surface that may contain Human Immunodeficiency Virus virions, including but not limited to the vagina in a woman, the oral cavity in a body, and the rectum in a body. It is a device intended to remove the threat of the infectious threat of Human Immunodeficiency Virus virions from body fluids. It is a device used to clean surfaces by removing the infectious threat of Human Immunodeficiency Virus present on such a surface or protect a body from becoming infected by the Human Immunodeficiency Virus carried by body fluids that might be deposited into the body by way of the vagina, the oral cavity, or the rectum. As the body fluids make contact with the filter medium any HIV virions present in the body fluids have the opportunity to engage the three cell surface receptors the CD 4 receptor, the CCR5 receptor and the CXCR4 receptor which are well known to the medical and scientific community due to the fact they appear naturally on the surface of the Human T-Helper cell. Since the HIV virion engaged cell-surface receptors located on the surface of the filter medium rather than located on the surface of an endogenous T-Helper cell inside the body, the infectious nature of the HIV virions is neutralized by either the HIV virion becoming trapped by the filter medium or the HIV virion is caused to harmlessly eject its genome. When HIV virions become trapped by the filter medium it is incapable of migrating further to successfully engaging endogenous T-Helper cells inside the body. When a HIV virion is caused to eject its genome, the HIV virion is incapable of infecting an endogenous T-Helper cell inside the body. Trapping the HIV virion or causing the HIV virion to harmlessly eject its genome leads to neutralizing the infectious threat of HIV, which leads to effectively averting AIDS.

### DETAILED DESCRIPTION OF THE INVENTION

The invention described herein is intended to neutralize the infectious nature of Human Immunodeficiency Virus virions carried in body fluids. The medical device is comprised of a filter medium or a filter medium suspended in a hypoallergenic fluid or cream. The medical device is intended to be applied to surfaces where body fluids infected with the Human Immunodeficiency Virus may be. The mixing of the filter medium comprising the medical device with fluids that contain Human Immunodeficiency Virus virions is meant to result in the neutralization of the infectious nature of the Human Immunodeficiency Virus virions.

Three cell receptors CD4, CCR5 and CXCR4 are well known to the medical and scientific community and appear naturally on the surface of the Human T-Helper cells. The HIV virion expresses gp 120 glycoprotein probes and glycoprotein 41 probes on its outer envelope. HIV utilizes the T-Helper cell as its host cell for the purposes of replication.

In completing the virus's natural reproductive-cycle, HIV utilizes the gp 120 probe positioned on the exterior envelope of an HIV virion to locate and engage a T-Helper cell's CD4 exterior cell-surface receptor. Once the HIV's gp 120 has successfully engaged a CD 4 cell-surface receptor, the HIV virion's gp 41 probe engages either a CCR5 or CXCR4 exterior cell-surface receptor on the T-Helper cell. A filter medium expressing CD4, CCR5 and CXCR4 cell-surface receptors offers a surrogate target expressing the cell-surface receptors HIV virions are seeking to engage. When HIV virion's probes encounter a filter medium expressing CD4, CCR5 and CXCR4 cell-surface receptors, affixed to the surface of the filter medium similar to the manner these cell-surface receptors are affixed to the surface of naturally occurring T-Helper cells, HIV's gp 120 probes would engage CD 4 exterior surface receptors, followed then by HIV's gp 41 probes engaging either CCR5 or CXCR4 exterior cell-surface receptors. Once the HIV gp 120 probes and gp 41 probes have engaged their respective cell-surface receptors affixed to the filter medium's exterior surface, the HIV is fixed to the surface of the filter medium and the HIV virion may eject RNA genome it carries. Since the HIV engaged a filter medium the HIV virion becomes affixed and trapped by the filter device and if the HIV virion ejects its RNA genome, the threat of the HIV virion being able to infect an endogenous T-Helper cell inside a body is effectively neutralized. The body fluids passing through such a filter becomes cleared of infectious HIV virions.

The medical device described herein, intended to neutralize infectious HIV virions in body fluids that come into contact with the filter medium. The filter medium comprising this medical device may be fashioned from a variety of different materials and fashioned in a variety of different shapes. The filter medium is intended to make available cell-surface receptors including CD 4, CCR5 and CXCR4, affixed to the surface of the filter medium similar to the manner these cell-surface receptors are affixed to the surface of naturally occurring T-Helper cells, for HIV virions to engage. The filter medium may be comprised of a quantity of exogenous T-Helper cells. The filter medium may be comprised of a quantity of lipid bilayer sheets which are comprised of similar materials as found existing as the outer membrane of a T-Helper cell, and affixed to the said lipid bilayer sheets are glycoprotein cell-surface receptors including a quantity of CD4 cell-surface receptors, CXCR4 cell-surface receptors, CCR5 cell-surface receptors. The filter medium may be comprised of a quantity of modified viruses or virus-like structures with cell-surface receptors to include a quantity of CD4 cell-surface receptors, CXCR4 cell-surface receptors, CCR5 cell-surface receptors. The filter medium may be comprised of any appropriate hypoallergenic material, which can be affixed to the surface a quantity of CD4 cell-surface receptors, CXCR4 cell-surface receptors, CCR5 cell-surface receptors or simply the protein portion of the CD4 cell-surface receptors, CXCR4 cell-surface receptors, CCR5 cell-surface receptors.

To carry out the process to manufacture a modified medically therapeutic virus, messenger RNA that would code for the general physical outer structures of the modified virus, such as instruction code to generate a modified HIV virion or a modified Hepatitis C virus virion or other virus virion or other virus-like structure, would be inserted into a host. The host may include devices such as a host cell or a hybrid host cell. The host may utilize DNA or RNA or a combination of genetic instructions in order to accomplish the construction of medically therapeutic modified virus virions. In some cases DNA or messenger RNA would be inserted into the host that would be coded to cause the production of surface probes that would be affixed to the surface of the virus virion that would target the glycoprotein probes affixed to the surface of an HIV virion. The copies of the medically therapeutic modified viruses or medically therapeutic virus-like structures, upon exiting the host, would be collected, stored and utilized as a medical treatment as necessary.

The medically therapeutic version of the modified virus and virus-like structures would be incapable of replication on its own due to the fact that the messenger RNA that would code for the replication process to produce copies of the virus or virus-like structure would not be present in the modified form of a virus or virus-like structure.

Lipid bilayer sheets can be manufactured and combinations of CD 4 cell-surface receptors, CXCR4 cell-surface receptors, CCR5 cell-surface receptors can be affixed to the surface similar to the manner these cell-surface receptors are affixed to the surface of naturally occurring T-Helper cells, with the entire structure acting as a filter medium. Sheets of any appropriate hypoallergenic material can be manufactured and combinations of CD 4 cell-surface receptors, CXCR4 cell-surface receptors, CCR5 cell-surface receptors can be affixed to the surface with the entire structure acting as a filter medium. Sheets of any appropriate hypoallergenic material can be manufactured and combinations of the protein portion of the CD 4 cell-surface receptors, CXCR4 cell-surface receptors, CCR5 cell-surface receptors available to engage either glycoprotein probes on HIV or cell-surface receptors on a T-Helper cell, affixed to the surface of the hypoallergenic material with the entire structure acting as a filter medium.

The filter medium may be suspended in any suitable hypoallergenic fluid or cream.

The filter medium may be mixed with and coexist with a quantity of an agent that acts as a spermatocide to prevent sperm present in the vagina from being able to proceed from the vagina to fertilize an egg and cause the woman to become pregnant.

The filter medium can be mixed and coexist with a quantity of lubricant.

The filter medium can be mixed and coexist with a quantity of moisturizer.

### DRAWINGS

None.

The terms and expressions which are employed here are used as terms of description and are not limitation and there is no intention, in the use of terms and expressions, of excluding equivalents of the features presented, and described, or portions thereof, it being recognized that various modifications are possible in the scope of the invention or process as claimed.

What is claimed:

1. A medical device to protect an individual from contracting the Human Immunodeficiency Virus comprised of:
(a) a filter medium,
(b) said filter medium having cell-surface receptors affixed to its surface,
whereby Human Immunodeficiency Virus virions are intended to come in contact with said cell-surface receptors found on the surface of said filter medium,
whereby said Human Immunodeficiency Virus virions are intended to engage said cell-surface receptors found on the surface of said filter medium with the intention of preventing said Human Immunodeficiency Virus virions from being able to infect T-Helper cells endogenous to a body by trapping the Human Immunodeficiency Virus virions on the filter medium or by neutralizing the infectious threat posed by said Human Immunodeficiency Virus virions by causing said Human Immunodeficiency Virus virions to harmlessly eject the genetic genome said Human Immunodeficiency Virus virions carry,
whereby the medical device can be applied to any surface to neutralize the threat an individual may face in regards to contracting an infection by the Human Immunodeficiency Virus,
whereby the medical device can be applied to any surface to act as a cleaning device to neutralize the threat an individual may face in regards to contracting an infection by the Human Immunodeficiency Virus,
whereby the medical device can be applied into the vagina of a woman to neutralize the threat to the individual of contracting an infection by the Human Immunodeficiency Virus,
whereby the medical device can be applied into the rectum of a body to neutralize the threat to the individual of contracting an infection by the Human Immunodeficiency Virus,
whereby the medical device can be applied to into the oral cavity of a body to neutralize the threat to the individual of contracting an infection by the Human Immunodeficiency Virus.

2. The medical device in claim 1 wherein said filter medium selected from the group consisting of a quantity of T-Helper cells, a quantity of lipid bilayer sheets, a quantity of lipid bilayer strips, a quantity of lipid bilayer spheres, a quantity of modified virus virions, a quantity of virus-like structures, a quantity of hypoallergenic surfaces fashioned in the shape of a sheet capable of supporting the functional expression of a quantity of cell-surface receptors, a quantity of hypoallergenic surfaces fashioned in the shape of a strip capable of supporting the functional expression of a quantity of cell-surface receptors, and a quantity of hypoallergenic surfaces fashioned in the shape of a sphere capable of supporting the functional expression of a quantity of cell-surface receptors,
whereby said filter medium acts as a surrogate target, due to the manner by which said cell-surface receptors affixed to the surface of said filter medium is in a manner similar to how CD4, CXCR4 and CCR5 cell-surface receptors are affixed to the surface of a naturally occurring T-Helper cell, so as said cell-surface receptors are able to attract and properly engage glycoprotein probes found on the surface of Human Immunodeficiency Virus virions.

3. The medical device in claim 1 wherein said filter medium is a quantity of T-Helper cells.

4. The medical device in claim 1 wherein said filter medium selected from the group consisting of a quantity of lipid bilayer sheets, a quantity of lipid bilayer strips, and a quantity of lipid bilayer spheres.

5. The medical device in claim 1 wherein said filter medium is a quantity of modified virus virions.

6. The medical device in claim 1 wherein said filter medium is a quantity of virus-like structures.

7. The medical device in claim 1 wherein said filter medium selected from the group consisting of a quantity of hypoallergenic surfaces fashioned in the shape of a sheet capable of supporting the functional expression of a quantity of cell-surface receptors, a quantity of hypoallergenic surfaces fashioned in the shape of a strip capable of supporting the functional expression of a quantity of cell-surface receptors, and a quantity of hypoallergenic surfaces fashioned in the shape of a sphere capable of supporting the functional expression of a quantity of cell-surface receptors.

8. The medical device in claim 1 wherein said cell-surface receptors selected from the group consisting of a quantity of CD4 cell-surface receptors, a quantity of CXCR4 cell-surface receptors and a quantity of CCR5 cell-surface receptors,
whereby said filter medium acts as a surrogate target, due to the manner by which said cell-surface receptors affixed to the surface of said filter medium is in a manner similar to how CD4, CXCR4 and CCR5 cell-surface receptors are affixed to the surface of a naturally occurring T-Helper cell, so as said cell-surface receptors are able to attract and properly engage glycoprotein probes found on the surface of Human Immunodeficiency Virus virions,
whereby, as on the surface of a T-Helper cell, a Human Immunodeficiency Virus virion glycoprotein 120 probe will engage a CD4 cell-surface receptor followed by a glycoprotein probe 41 probe on said Human Immunodeficiency Virus virion engaging either a CXCR4 cell surface receptor or a CCR5 cell-surface receptor.

9. The medical device in claim 1 wherein said cell-surface receptors consist of a quantity of CD4 cell-surface receptors, a quantity of CXCR4 cell-surface receptors and a quantity of CCR5 cell-surface receptors,
whereby said filter medium acts as a surrogate target, due to the manner by which said cell-surface receptors affixed to the surface of said filter medium is in a manner similar to how CD4, CXCR4 and CCR5 cell-surface receptors are affixed to the surface of a naturally occurring T-Helper cell, so as said cell-surface receptors are able to attract and properly engage glycoprotein probes found on the surface of Human Immunodeficiency Virus virions,
whereby, as on the surface of a T-Helper cell, a Human Immunodeficiency Virus virion glycoprotein 120 probe will engage a CD4 cell-surface receptor followed by a glycoprotein probe 41 probe on said Human Immunodeficiency Virus virion engaging either a CXCR4 cell surface receptor or a CCR5 cell-surface receptor.

10. The medical device in claim 1 wherein said filter medium may be suspended in a hypoallergenic fluid.

11. A medical device to protect an individual from contracting the Human Immunodeficiency Virus comprised of a:
(a) a filter medium,
(b) said filter medium having cell-surface receptors affixed to its surface,
(c) said filter medium is suspended in a hypoallergenic fluid,
whereby Human Immunodeficiency Virus virions are intended to come in contact with said cell-surface receptors found on the surface of said filter medium,
whereby said Human Immunodeficiency Virus virions are intended to engage said cell-surface receptors found on the surface of said filter medium with the intention of preventing said Human Immunodeficiency Virus virions from being able to infect T-Helper cells endogenous to a body by trapping the Human Immunodeficiency Virus virions on the filter medium or by neutralizing the infectious threat posed by said Human Immunodeficiency Virus virions by causing said Human Immunodeficiency Virus virions to harmlessly eject the genetic genome said Human Immunodeficiency Virus virions carry,
whereby the medical device can be applied to any surface to neutralize the threat to an individual of contracting an infection by the Human Immunodeficiency Virus,
whereby the medical device can be applied to any surface to act as a cleaning device to neutralize the threat to an individual of contracting an infection by the Human Immunodeficiency Virus,
whereby the medical device can be applied into the vagina of a woman to neutralize the threat to said woman of contracting an infection by the Human Immunodeficiency Virus,
whereby the medical device can be applied into the rectum of a body to neutralize the threat to the individual of contracting an infection by the Human Immunodeficiency Virus,
whereby the medical device can be applied to into the oral cavity of a body to neutralize the threat to the individual of contracting an infection by the Human Immunodeficiency Virus.

12. The medical device in claim 11 wherein said filter medium selected from the group consisting of a quantity of T-Helper cells, a quantity of lipid bilayer sheets, a quantity of lipid bilayer strips, a quantity of lipid bilayer spheres, a quantity of modified virus virions, a quantity of virus-like structures, a quantity of hypoallergenic surfaces fashioned in the shape of a sheet capable of supporting the functional expression of a quantity of cell-surface receptors, a quantity of hypoallergenic surfaces fashioned in the shape of a strip capable of supporting the functional expression of a quantity of cell-surface receptors, and a quantity of hypoallergenic surfaces fashioned in the shape of a sphere capable of supporting the functional expression of a quantity of cell-surface receptors,
whereby said filter medium acts as a surrogate target, due to the manner by which said cell-surface receptors affixed to the surface of said filter medium is in a manner similar to how CD4, CXCR4 and CCR5 cell-surface receptors are affixed to the surface of a naturally occurring T-Helper cell, so as said cell-surface receptors are able to attract and properly engage glycoprotein probes found on the surface of Human Immunodeficiency Virus virions.

13. The medical device in claim 11 wherein said filter medium is a quantity of T-Helper cells.

14. The medical device in claim 11 wherein said filter medium selected from the group consisting of a quantity of lipid bilayer sheets, a quantity of lipid bilayer strips, and a quantity of lipid bilayer spheres.

15. The medical device in claim 11 wherein said filter medium is a quantity of modified virus virions.

16. The medical device in claim 11 wherein said filter medium is a quantity of virus-like structures.

17. The medical device in claim 11 wherein said filter medium selected from the group consisting of a quantity of hypoallergenic surfaces fashioned in the shape of a sheet capable of supporting the functional expression of a quantity of cell-surface receptors, a quantity of hypoallergenic surfaces fashioned in the shape of a strip capable of supporting the functional expression of a quantity of cell-surface receptors, and a quantity of hypoallergenic surfaces fashioned in the shape of a sphere capable of supporting the functional expression of a quantity of cell-surface receptors.

18. The medical device in claim 11 wherein said cell-surface receptors selected from the group consisting of a quantity of CD4 cell-surface receptors, a quantity of CXCR4 cell-surface receptors and a quantity of CCR5 cell-surface receptors,
whereby said filter medium acts as a surrogate target, due to the manner by which said cell-surface receptors affixed to the surface of said filter medium is in a manner similar to how CD4, CXCR4 and CCR5 cell-surface receptors are affixed to the surface of a naturally occurring T-Helper cell, so as said cell-surface receptors are able to attract and properly engage glycoprotein probes found on the surface of Human Immunodeficiency Virus virions,
whereby, as on the surface of a T-Helper cell, a Human Immunodeficiency Virus virion glycoprotein 120 probe will engage a CD4 cell-surface receptor followed by a glycoprotein probe 41 probe on said Human Immunodeficiency Virus virion engaging either a CXCR4 cell surface receptor or a CCR5 cell-surface receptor.

19. The medical device in claim 11 wherein said cell-surface receptors consist of a quantity of CD4 cell-surface receptors, a quantity of CXCR4 cell-surface receptors and a quantity of CCR5 cell-surface receptors,
whereby said filter medium acts as a surrogate target, due to the manner by which said cell-surface receptors affixed to the surface of said filter medium is in a manner similar to how CD4, CXCR4 and CCR5 cell-surface receptors are affixed to the surface of a naturally occurring T-Helper cell, so as said cell-surface receptors are able to attract and properly engage glycoprotein probes found on the surface of Human Immunodeficiency Virus virions,
whereby, as on the surface of a T-Helper cell, a Human Immunodeficiency Virus virion glycoprotein 120 probe will engage a CD4 cell-surface receptor followed by a glycoprotein probe 41 probe on said Human Immunodeficiency Virus virion engaging either a CXCR4 cell surface receptor or a CCR5 cell-surface receptor.

20. The medical device in claim 11 wherein said filter medium can be mixed with other fluids such that the filter medium may coexist with other fluid components selected from the group consisting of a quantity of lubricant, a quantity of moisturizer, and a quantity of spermicidal agent.
